(19) United States
(12) Patent Application Publication
(10) Pub. No.: US 2010/0256761 A1
(43) Pub. Date: Oct. 7, 2010

(54) MINIMALLY INVASIVE TOTAL SPINE IMPLANT

(76) Inventors: Richard D. Komistek, Knoxville, TN (US); Mohamed R. Mahfouz, Knoxville, TN (US); Joseph S. Cheng, Nashville, TN (US)

Correspondence Address: TAFT, STETTINIUS & HOLLISTER LLP, SUITE 1800, 425 WALNUT STREET, CINCINNATI, OH, 45202-3957, US

(21) Appl. No.: 12/417,950

(22) Filed: Apr. 3, 2009

(51) Int. Cl. A61F 2/44

(52) U.S. Cl. 623/17.16; 623/17.11

(57) ABSTRACT

A spinal disc prosthesis comprising: (a) a distal spinal prosthetic comprising at least two separable portions; (b) a proximal spinal prosthetic comprising at least two separable portions, where at least one of the two separable portions of at least one of the distal spinal prosthetic and the proximal spinal prosthetic includes a partial loop appendage at least partially defining a spinal nerve canal.

### RELATED ART

1. Field of the Disclosure

The present disclosure relates to orthopaedic implants and, specifically, to spinal implants for use in joint replacement and revision procedures in the treatment of spinal pathologies. The disclosure also pertains to the development of an artificial spinal joint that is designed to be inserted from a minimally invasive approach to the spine.

2. Brief Discussion of Related Art

At present, spinal surgery has become the most rapidly growing area in orthopaedics. Previously, patients having disc degeneration would undergo a spinal fusion to offset pain in the neck or lower back. More recently, artificial discs have been developed to replace degenerated discs and facet replacements have been introduced to replace failing facets. Unfortunately, disc replacements have not yet proven to be successful and facet replacements are in their infancy. Most facet replacements are performed using an investigational device exemption (IDE) that includes removal of present facet bones and replacement by a metal facet that is screwed into the vertebral bones. It is hypothesized that these types of replacements will fail dramatically, since the vertebral bodies do not have extensive bone stock and induced torques will loosen these devices.

Disc replacements and facet replacements are also implanted through an “open” technique, which requires making a large incision along the middle of the back or along the abdomen. The surgeon then cuts and pulls back spinal muscles and tissue to get to the vertebrae and disc space in order to position the implanted devices. These surgical techniques can cause tissue trauma and damage due to muscle, ligament, tendon, bone, and local nerve injury from performing this surgical procedure. This may lead the body to biomechanically rely more on the facet replacements, thereby affecting facet stability. In contrast, the use of minimally invasive surgical techniques has been hypothesized to reduce these negative consequences.

### INTRODUCTION TO THE INVENTION

The instant disclosure includes a total spine replacement optionally having both disc and facet replacement using a total joint-type procedure. Initially, minimal bone is resected distally from the upper vertebral body and proximally from the distal vertebral body. Then, the bearing surfaces of the facets are removed from the bone. A distal disc replacement is mounted to the upper vertebral body and a proximal disc replacement is mounted to the lower vertebral body. Both of these disc replacement devices may be multi-piece implants that join together, optionally in the center. Polyethylene materials (such as for bearing surfaces), or other known or developed materials, may then mounted to the proximal and distal devices to replace the degenerated disc. The replacement disc material may be mounted in a fixed orientation or may exhibit mobile bearing characteristics. Polyethylene inserts, or another known or developed material, may also be rigidly attached to the facets as facet replacements. Accordingly, the exemplary embodiments may be utilized to replace one or both of the disc and the facets.

It is a first aspect of the present invention to provide a spinal disc prosthesis comprising: (a) a distal spinal prosthetic comprising at least two separable portions; and (b) a proximal spinal prosthetic comprising at least two separable portions, where at least one of the two separable portions of at least one of the distal spinal prosthetic and the proximal spinal prosthetic includes a partial loop appendage at least partially defining a spinal nerve canal.

In a more detailed embodiment of the first aspect, the distal spinal prosthetic includes a first bearing insert mounted to at least one of the at least two separable portions, the first bearing insert including a bearing surface interposing the distal spinal prosthetic and the proximal spinal prosthetic. In yet another more detailed embodiment, at least one of the complementary halves includes a cavity for receiving a projection of the first bearing insert to mount the first bearing insert to at least one of the at least two separable portions. In a further detailed embodiment, at least one of the complementary halves includes a projection received within a cavity of the first bearing insert to mount the first bearing insert to at least one of the at least two separable portions. In still a further detailed embodiment, the partial loop appendage extends from a rear surface of at least one of the distal spinal prosthetic and the proximal spinal prosthetic. In a more detailed embodiment, the partial loop appendage extends from a top surface of at least one of the distal spinal prosthetic and the proximal spinal prosthetic. In a more detailed embodiment, the partial loop appendage extends from a bottom surface of at least one of the distal spinal prosthetic and the proximal spinal prosthetic. In another more detailed embodiment, the partial loop appendage includes a helical portion. In yet another more detailed embodiment, the partial loop appendage extends from a side surface of at least one of the distal spinal prosthetic and the proximal spinal prosthetic. In still another more detailed embodiment, both the distal spinal prosthetic and the proximal spinal prosthetic include at least one partial loop appendage at least partially defining the spinal nerve canal.

In yet another more detailed embodiment of the first aspect, both the distal spinal prosthetic and the proximal spinal prosthetic include a pair of partial loop appendages at least partially defining the spinal nerve canal. In still another more detailed embodiment, the pair of partial loop appendages of the proximal spinal prosthetic includes an arcuate diameter less than an arcuate diameter of the pair of partial loop appendages of the distal spinal prosthetic. In a further detailed embodiment, interior surfaces of the pair of partial loop appendages of the distal spinal prosthetic provides camming surfaces for exterior surfaces of the pair of partial loop appendages of the proximal spinal prosthetic. In still a further detailed embodiment, the distal spinal prosthetic includes a pair of partial loop appendages at least partially defining the spinal nerve canal. In a more detailed embodiment, the proximally spinal prosthetic includes a pair of partial loop appendages at least partially defining the spinal nerve canal. In a more detailed embodiment, the partial loop appendage includes a facet replacement. In another more detailed embodiment, the proximal spinal prosthetic includes a first bearing insert mounted to at least one of the at least two separable portions, the first bearing insert including a bearing surface interposing the distal spinal prosthetic and the proximal spinal prosthetic. In yet another more detailed embodiment, at least one of the complementary halves includes a cavity for receiving a projection of the first bearing insert to mount the first bearing insert to at least one of the at least two separable portions.

In yet a further detailed embodiment of the first aspect, at least one of the complementary halves includes a projection received within a cavity of the first bearing insert to mount the first bearing insert to at least one of the at least two separable portions. In still another more detailed embodiment, at least one of the distal spinal prosthetic and the proximal spinal prosthetic includes a twisted appendage including a facet interface pad. In a further detailed embodiment, the first bearing insert is a fixed bearing insert with respect to at least one of the at least two separable portions. In still a further detailed embodiment, the first bearing insert is a mobile bearing insert with respect to at least one of the at least two separable portions.

It is a second aspect of the present invention to provide an intervertebral prosthetic comprising a first spinal implant including a vertebral body portion, the vertebral body portion at least partially replicating the horizontal cross-section of a vertebral body and including a vertebral contacting surface adapted to contact a native vertebral body, the first spinal implant also including a disc portion including a bearing surface, the first spinal implant also including an appendage at least partially defining a spinal nerve conduit.

In a more detailed embodiment of the second aspect, the first implant includes a pair of appendages at least partially defining a spinal nerve conduit. In yet another more detailed embodiment, the appendage includes a helical portion. In a further detailed embodiment, the appendage includes a twisted portion. In still a further detailed embodiment, the appendage includes a facet replacement portion. In a more detailed embodiment, the vertebral body portion is a separable component from the disc portion. In a more detailed embodiment, the vertebral body portion is fabricated from a metal and the disc portion is fabricated from a polymer. In another more detailed embodiment, the vertebral body portion includes a cavity that receives a projection of the disc portion to mount the vertebral body portion to the disc portion. In yet another more detailed embodiment, the disc portion includes a cavity that receives a projection of the vertebral body portion to mount the vertebral body portion to the disc portion. In still another more detailed embodiment, the vertebral body portion is at least one of glued and cemented to the disc portion.

It is a third aspect of the present invention to provide an intervertebral implant comprising: (a) a first implant adapted to mate with a first vertebra, where the first implant includes an arm operative to at least partially define a spinal nerve passage; and (b) a second implant adapted to mate with a second vertebra consecutive to the first vertebra, where the second implant includes an appendage operative to cooperate with the arm of the first implant to at least partially define the spinal nerve passage.

In a more detailed embodiment of the third aspect, the first implant includes a first bearing insert having a first bearing surface interposing the first vertebra and the second vertebra. In yet another more detailed embodiment, the second implant includes a second bearing insert having a second bearing surface interposing the first vertebra and the second vertebra, the second bearing surface adapted to interface with the first bearing surface of the first implant. In a further detailed embodiment, the first implant comprises a first vertebral segment and a first disc segment mounted to one another. In still a further detailed embodiment, the first vertebral segment includes a cavity receiving a projection from the first disc segment to mount the first vertebral segment to the first disc segment. In a more detailed embodiment, the first disc segment includes a cavity receiving a projection from the first vertebral segment to mount the first vertebral segment to the first disc segment. In a more detailed embodiment, the first implant comprises at least two component parts simulating the horizontal cross-section of a vertebral body. In another more detailed embodiment, the at least two component parts comprise complementary halves of a base plate. In yet another more detailed embodiment, the base plate halves are mounted to a bearing insert. In still another more detailed embodiment, the second implant comprises a second vertebral segment and a second disc segment mounted to one another.

In yet another more detailed embodiment of the third aspect, the first vertebral segment includes a cavity receiving a projection from the first disc segment to mount the first vertebral segment to the first disc segment. In still another more detailed embodiment, the first disc segment includes a cavity receiving a projection from the first vertebral segment to mount the first vertebral segment to the first disc segment. In a further detailed embodiment, the second implant comprises at least two component parts simulating the horizontal cross-section of a vertebral body. In still a further detailed embodiment, the at least two component parts comprise complementary halves of a base plate. In a more detailed embodiment, the base plate halves are adapted to receive a bearing insert. In a more detailed embodiment, the arm of the first implant extends from a rear surface a base plate at least partially simulating the horizontal cross-section of a vertebral body. In another more detailed embodiment, the arm of the first implant extends from a top surface a base plate at least partially simulating the horizontal cross-section of a vertebral body. In yet another more detailed embodiment, the arm of the first implant extends from a bottom surface a base plate at least partially simulating the horizontal cross-section of a vertebral body.

In yet further more detailed embodiment of the third aspect, the arm of the first implant extends from a side surface a base plate at least partially simulating the horizontal cross-section of a vertebral body. In still another more detailed embodiment, the arm of the first implant includes a helical portion. In a further detailed embodiment, the appendage of the second implant extends from a rear surface a base plate at least partially simulating the horizontal cross-section of a vertebral body. In still a further detailed embodiment, the appendage of the second implant extends from a top surface a base plate at least partially simulating the horizontal cross-section of a vertebral body. In a more detailed embodiment, the appendage of the second implant extends from a bottom surface a base plate at least partially simulating the horizontal cross-section of a vertebral body. In a more detailed embodiment, the appendage of the second implant extends from a side surface a base plate at least partially simulating the horizontal cross-section of a vertebral body. In another more detailed embodiment, the appendage of the second implant includes a helical portion. In yet another more detailed embodiment, the first implant includes a pair of arms operative to at least partially define a pair of spinal nerve passages, and the second implant includes a pair of appendages operative to at least partially define the pair of spinal nerve passages.

In still a further more detailed embodiment of the third aspect, each of the pair of arms includes at least one of a twisted portion and a helical portion, and each of the pair of appendages includes at least one of a twisted portion and a helical portion. In still another more detailed embodiment, each of the pair of arms includes a helical portion, each of the pair of appendages includes a helical portion, and each of the pair of arms of the first implant includes an arcuate diameter less than an arcuate diameter of the pair of appendages of the second implant. In a further detailed embodiment, the arm includes a facet replacement. In still a further detailed embodiment, the appendage includes a facet replacement.

It is a fourth aspect of the present invention to provide a spinal implant comprising: (a) a first platform allowing fixation to a first vertebra; (b) a second platform allowing fixation to a second vertebra immediately following the first vertebra; and (c) a bearing interposing the first platform and the second platform, where at least one of the first platform, the second platform, and the bearing includes an arcuate projection at least partially defining a spinal nerve passageway.

It is a fifth aspect of the present invention to provide a spinal disc prosthesis comprising: (a) a distal spinal prosthetic comprising a distal base plate and a distal bearing; and (b) a proximal spinal prosthetic comprising a proximal base plate and a proximal bearing, where at least one of the distal spinal prosthetic and the proximal spinal prosthetic includes an appendage at least partially defining a spinal nerve canal.

### BRIEF DESCRIPTION OF THE DRAWINGS

FIG. 1 is a profile view of an exemplary human spinal column.

FIG. 2 is an overhead view of a lumber vertebrae.

FIG. 3 is a profile view of a pair of lumbar vertebrae.

FIG. 4 is an overhead view of a pair of lumbar vertebrae interposed by a first exemplary spinal implant embodiment of the instant disclosure.

FIG. 5 a left profile view of the first exemplary spinal implant embodiment between the consecutive lumbar vertebrae of FIG. 4.

FIG. 6 an elevated perspective view from the left front of the first exemplary spinal implant embodiment between the consecutive lumbar vertebrae of FIG. 4.

FIG. 7 a frontal view of the first exemplary spinal implant embodiment between the consecutive lumbar vertebrae of FIG. 4.

FIG. 8 an elevated perspective view from the right front of the first exemplary spinal implant embodiment between the consecutive lumbar vertebrae of a human body.

FIG. 9 is an elevated perspective view from the right rear of the first exemplary spinal implant embodiment between the consecutive lumbar vertebrae of a human body.

FIG. 10 is a bottom view of an exemplary proximal component part of the first exemplary spinal implant embodiment.

FIG. 11 is a top view of an exemplary proximal component part of the first exemplary spinal implant embodiment.

FIG. 12 is a bottom view of exemplary proximal base plate halves, separated, of the first exemplary spinal implant embodiment.

FIG. 13 is a bottom view of the exemplary proximal base plate halves of FIG. 12, joined.

FIG. 14 is a bottom view of exemplary proximal insert halves, separated, of the first exemplary spinal implant embodiment.

FIG. 15 is a right profile view of the exemplary proximal insert halves of FIG. 14, joined.

FIG. 16 a top view of an exemplary distal component part of the first exemplary spinal implant embodiment.

FIG. 17 is a top view of exemplary distal base plate halves, joined, of the first exemplary spinal implant embodiment.

FIG. 18 is a top view of exemplary distal base plate halves, separated, of the first exemplary spinal implant embodiment.

FIG. 19 is a top view of exemplary distal insert halves, separated, of the first exemplary spinal implant embodiment.

FIG. 20 is a left profile view of the exemplary distal insert halves of FIG. 19, joined.

FIG. 21 is a left profile view of alternate exemplary distal insert halves, joined.

FIG. 22 is a frontal view of the alternate exemplary distal insert halves of FIG. 21.

FIG. 23 is a top view of an alternate exemplary distal base plate.

FIG. 24 is a top view of a further alternate exemplary distal base plate.

FIG. 25 is a left profile view of an alternate exemplary distal component.

FIG. 26 is a right profile view of two consecutive lumbar vertebrae.

FIG. 27 is a right profile view of the two consecutive lumbar vertebrae of FIG. 26 being notched out to accept a spinal implant.

FIG. 28 is a bottom right lowered perspective view showing the exemplary positioning of a proximal base plate right half with respect to a proximal lumbar vertebra.

FIG. 29 is a bottom left lowered perspective view showing the exemplary positioning of the proximal base plate right half of FIG. 28 with respect to both proximal and distal lumbar vertebrae.

FIG. 30 is a bottom right lowered perspective view showing the exemplary positioning of a proximal base plate left, half with respect to both proximal and distal lumbar vertebrae.

FIG. 31 is a right profile view showing the exemplary positioning of proximal base plate halves with respect to both proximal and distal lumbar vertebrae.

FIG. 32 is an elevated perspective view from the left rear showing the exemplary positioning of a distal base plate right half with respect to a distal lumbar vertebra.

FIG. 33 is a right profile view showing the exemplary positioning of proximal and distal base plate halves with respect to both proximal and distal lumbar vertebrae.

FIG. 34 is a right profile view showing the exemplary positioning of proximal and distal base plate halves, as well a proximal insert halves, with respect to both proximal and distal lumbar vertebrae.

FIG. 35 is a right profile view showing the exemplary positioning of proximal and distal base plate halves, as well a proximal and distal insert halves, with respect to both proximal and distal lumbar vertebrae.

FIG. 36 is an elevated perspective view from the right rear showing the exemplary positioning of proximal and distal base plate halves, as well a proximal and distal insert halves, with respect to both proximal and distal lumbar vertebrae.

FIG. 37 is a right profile view showing the exemplary positioning of proximal and distal base plate halves, as well a proximal and distal insert halves, with respect to both proximal and distal lumbar vertebrae as well as the spinal column nerves extending through a right side opening defined by the spinal implant.

FIG. 38 is an elevated perspective view from the right front showing the exemplary positioning of proximal and distal base plate halves, as well a proximal and distal insert halves, with respect to both proximal and distal lumbar vertebrae as well as the spinal column nerves extending through a right side opening defined by the spinal implant.

### DETAILED DESCRIPTION

The exemplary embodiments of the present disclosure are described and illustrated below to encompass spinal prosthetics and associated methods of fabricating and implanting spinal prosthetics. Of course, it will be apparent to those of ordinary skill in the art that the embodiments discussed below are exemplary in nature and may be reconfigured without departing from the scope and spirit of the present disclosure. However, for clarity and precision, the exemplary embodiments as discussed below may include optional steps, methods, and features that one of ordinary skill should recognize as not being a requisite to fall within the scope of the present disclosure. For example, while the exemplary embodiments are described with respect to lumbar spinal implants, those skilled in the art will understand that the following aspects of the disclosure encompass all spinal implants, including, but not limited to, cervical spinal implants.

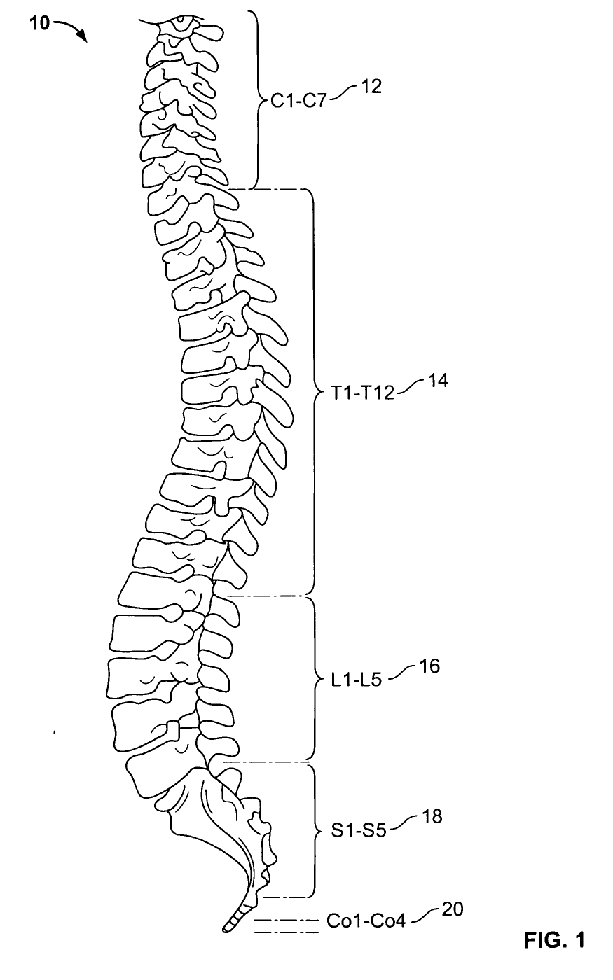

As shown in FIG. 1, the human spinal column 10 is comprised of a series of thirty-three stacked vertebrae divided into five regions. The cervical region includes seven vertebrae 12, known as C₁-C₇, the thoracic region includes twelve vertebrae 14, known at T₁-T₁₂, the lumbar region contains five vertebrae 16, known as L₁-L₅, the sacral region is comprised of five vertebrae 18, known as S₁-S₅, and the coccygeal region contains four vertebrae 20, known as Co₁-Co₄.

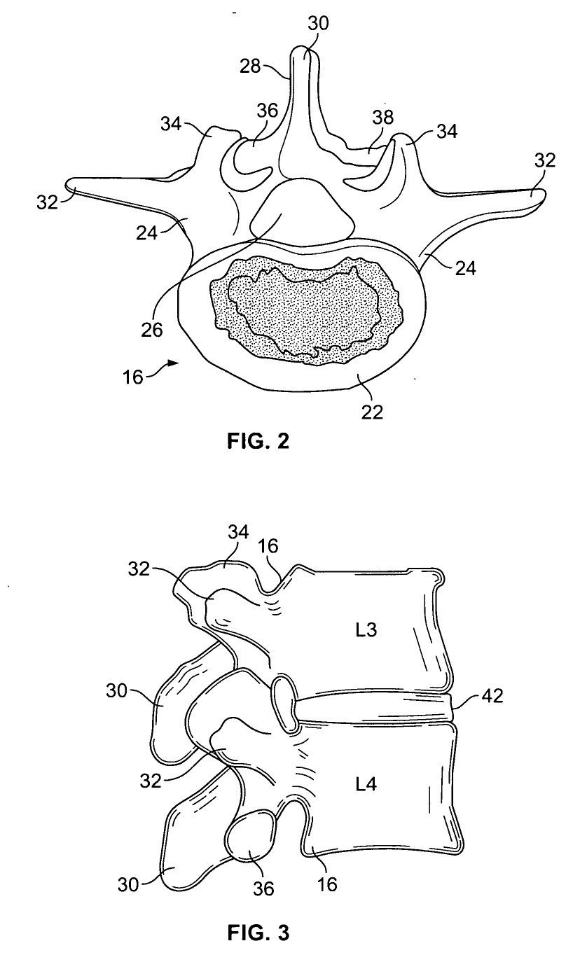

FIG. 2 shows an exemplary lumbar vertebra 16. Although lumbar vertebrae 16 vary somewhat according to location, these vertebrae share many features in common with most other vertebrae. Each vertebra 16 includes a vertebral body 22 and pedicles 24 that extend from each side of the vertebral body 22 to form a vertebral arch 26. At the posterior end of each pedicle 24, the vertebral arch 26 transitions into laminae 28. The laminae 28 fuse with each other to form a spinous process 30 that allow for muscle and ligamentous attachment.

Two transverse processes 32 thrust laterally outward from each side of the junction of the pedicle 24 with the lamina 28. The transverse processes 32 serve as levers for the attachment of muscles to the vertebrae 16. Four articular processes, two superior 34 and two inferior 36, also rise from the junctions of the pedicles 24 and the laminae 28. The superior articular processes 34 are sharp oval plates of bone rising upward on each side from the union of the pedicle 24 with the lamina 28. The inferior processes 36 are oval plates of bone that extend in an inferior direction on each side. Both processes 34, 36 include facets, with the superior articular facet 38 facing upward or superiorly, while the inferior articular facet faces downward or inferiorly.

As shown in FIG. 3, when adjacent lumbar vertebrae 16 are aligned, the facets 38, which are capped with smooth articular cartilage, interlock to form a facet joint, commonly referred to as a zygapophysial joint. The facet joint is composed of a superior half and an inferior half. The superior half is formed by the vertebral level below the joint, and the inferior half is formed by the vertebral level above the joint. For example, in the L₃-L₄ facet joint, the superior portion of the joint is formed by a bony structure on the L₄ vertebra (e.g., a superior articular surface and supporting bone on the L₄ vertebra), and the inferior portion of the joint is formed by a bony structure on the L₃ vertebra (e.g., an inferior articular surface and supporting bone on the L₃ vertebra). As also shown in FIG. 3, an intervertebral disc 42 located between each pair of vertebrae 16 permits relative movement between the vertebral bodies 22. Thus, the structure and alignment of the vertebrae 16 permit a range of movement relative to each other.

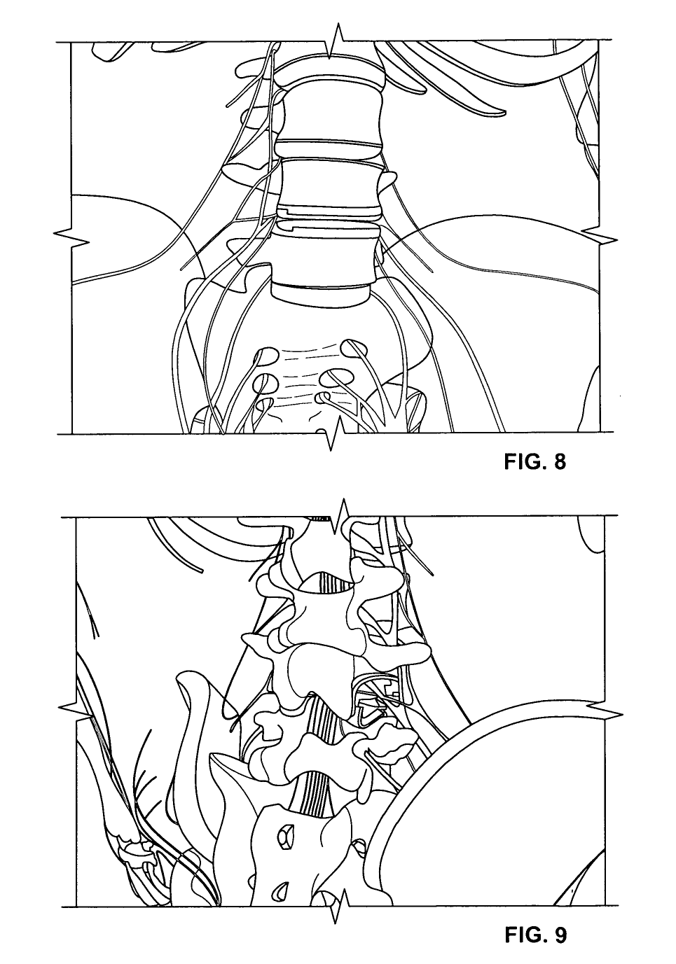

Referencing FIGS. 4-9, a first exemplary lumbar spinal implant 100 includes a proximal component 102 and a complimentary distal component 104 cooperating to provide a lumbar disc replacement prosthetic. The proximal component 102 includes a two-piece base plate 106, 108 and a two-piece insert 110, 112 mounted to the base plate 106, 108. In exemplary from, the base plate comprises substantially mirror image halves 106, 108 that are joinable by way of a locking system. It should be understood, however, that the proximal component 102 and the distal component 104 may each comprise a single part or a compilation of three or more complimentary parts.

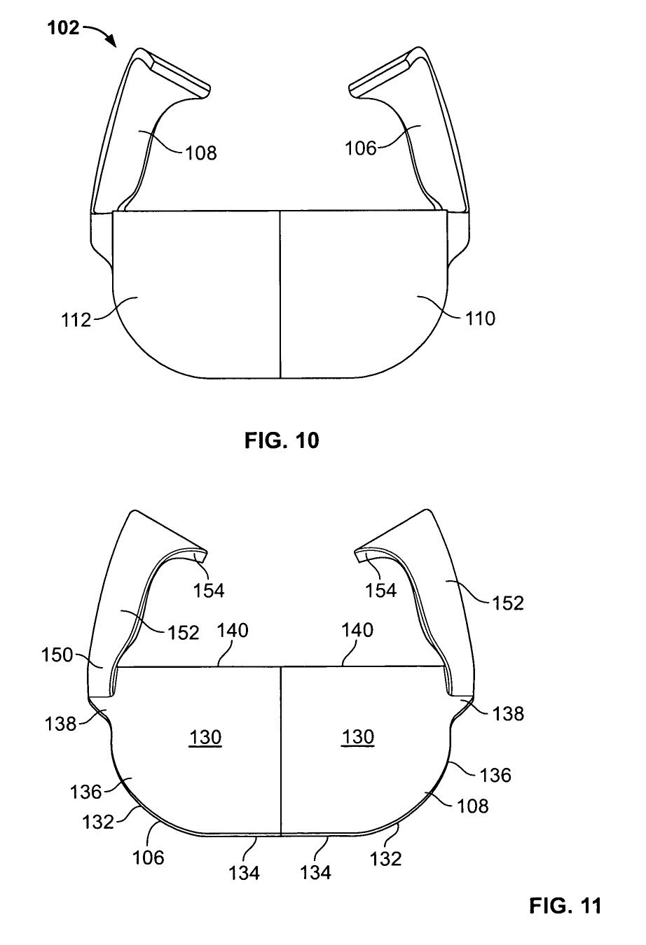

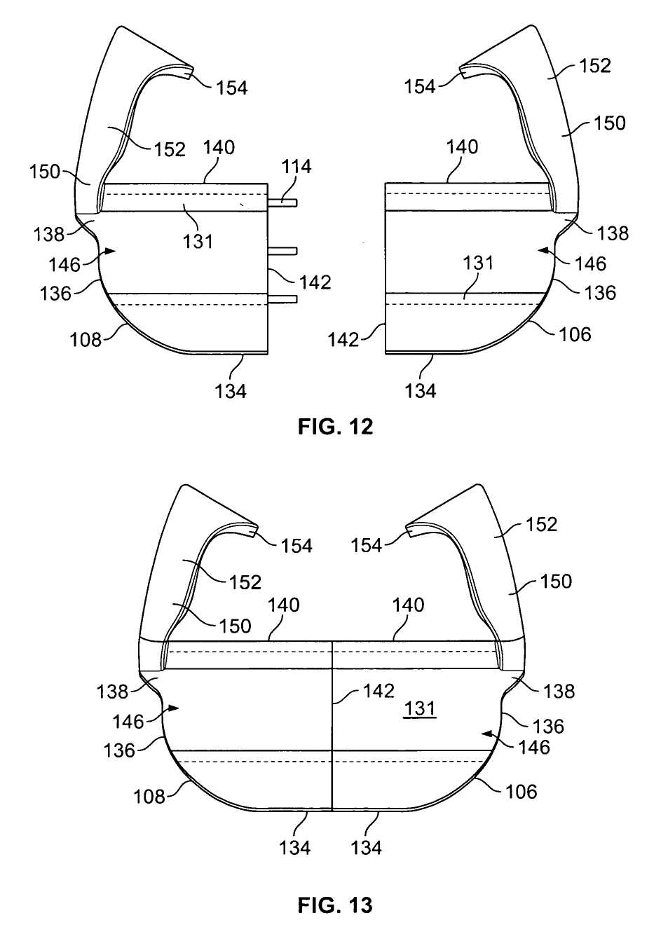

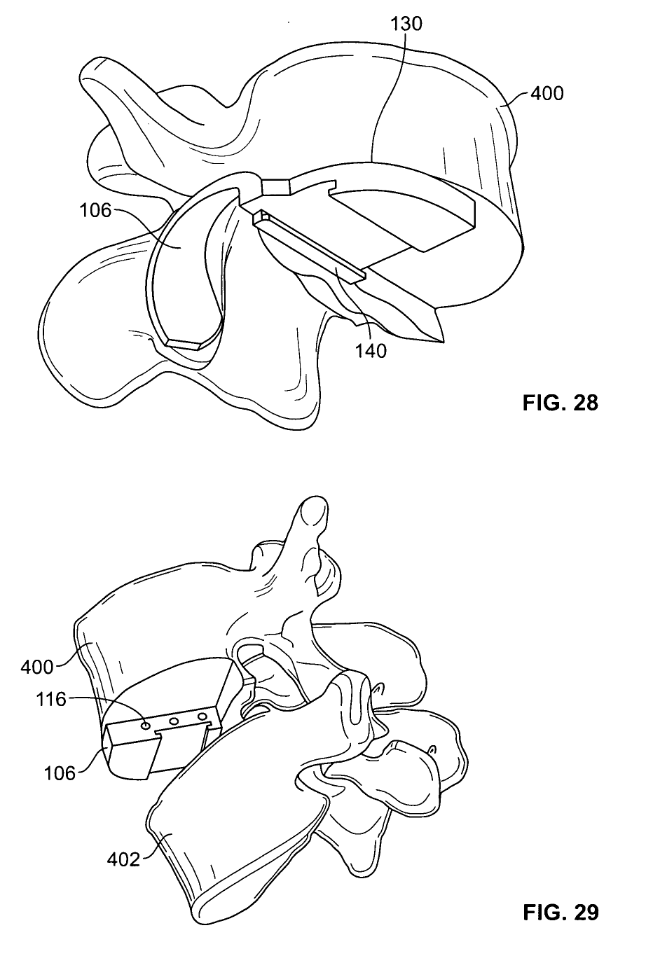

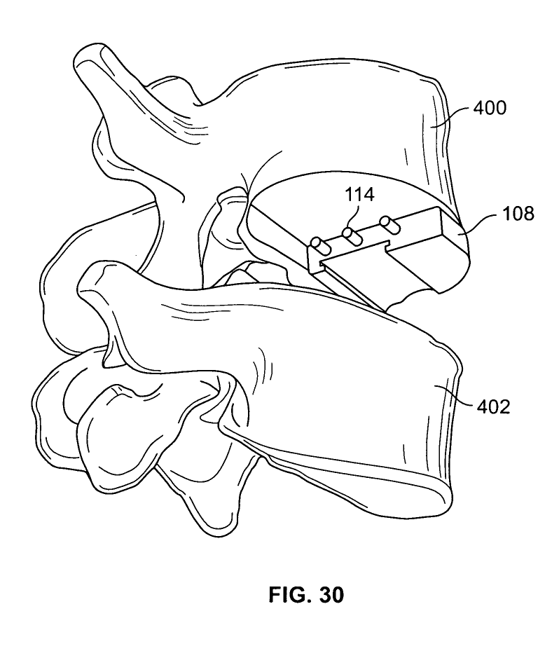

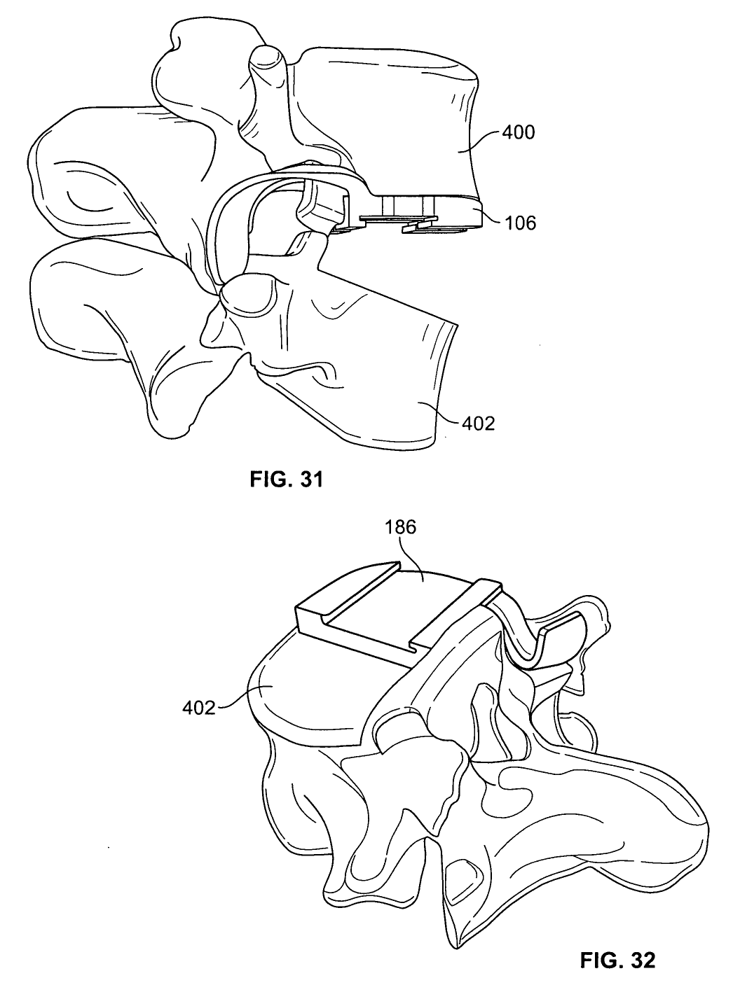

Referring to FIGS. 10-13, each proximal plate half 106, 108 includes a substantially planar top surface 130 that is generally operative to cross-sectionally duplicate the vertebral body 22 (See e.g., FIG. 28) against which it is mounted. As will be discussed hereafter, the vertebral body 22 is resurfaced in preparation for receiving the proximal plate halves 106, 108. For purposes of explanation only, each plate half 106, 108 includes a continuous perimeter 132 comprising a front face 134 arcuately transitioning to a side face 136, with a projecting ledge 138, that transitions into a rear face 140. Both the front face 134 and the rear face 140 transition at generally a right angle to an engagement face 142, where the engagement face 142 of each plate half 106, 108 abuts the other. A bottom surface 131 of each plate half 106, 108 includes a lateral groove 146 for receiving a projection 160 (see FIG. 15) from one of the proximal inserts 110, 112. In this exemplary embodiment, the groove 146 exhibits an inverted T-shape cross-section to receive the T-shape projection 160 of the inserts 110, 112. It is also within the scope of the disclosure to utilize other shaped grooves such as, without limitation, dove-tail grooves, block grooves, and tapered grooves.

Each proximal plate half 106, 108 also includes an arm 150 proximally extending from the projecting ledge 138. Each arm 150 proximate the ledge 138 includes a rectangular cross-section (having front, back, and opposing sides), that transitions into a hood-shape with the width of the sides decreasing and the width of the front and rear sides increasing. The arms 150, extending from the ledges 138, exhibit a mild spiral 152 that terminates at a rounded end 154. The spiral 152 arcs rearward (away from the rear face 140) and inward (away from the side face 136) to terminate proximate the inferior processes 36 of the vertebrae to which it is mounted. The spiral 152 is adapted to at least partially define an opening for egress of nerves exiting the spinal column between the vertebrae. As will be discussed in more detail hereafter, the distal component 104 also include arms that cooperate with the arms 150 of the proximal component 102 to define opposing lateral openings through which nerves exiting the spinal column may pass between the vertebrae.

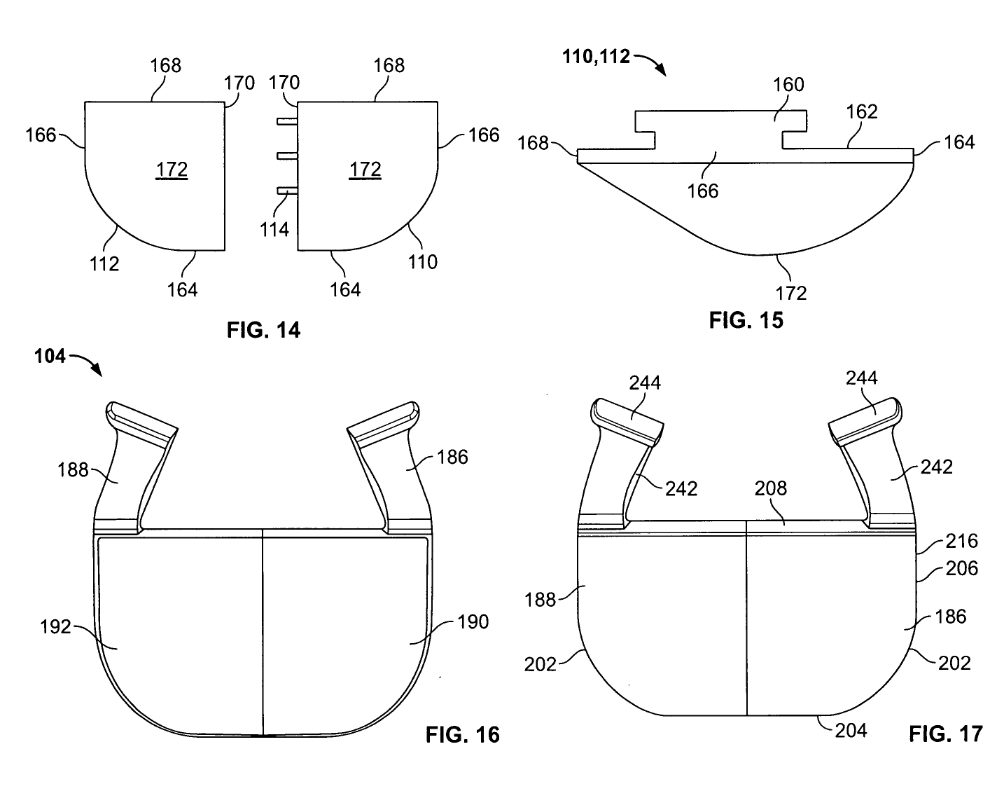

Referring to FIGS. 14 and 15, each proximal insert half 110, 112 cooperates to provide a flexible bushing interposing the plate halves 106, 108 and the distal component 104. As discussed briefly beforehand, each insert half 110, 112 includes a T-shape projection 160 that is received within a corresponding groove 146 of one of the plate halves 106, 108. This projection 160 extends from a substantially planar top surface 162 that abuts a bottom surface 131 of each half 106, 108. Depending upon the overall size and shape of the insert halves 110, 112, each half may exhibit a front face 164, arcuately transitioning to a side face 166 that transitions into a rear face 168. Both the front face 164 and the rear face 168 transition at generally a right angle to an engagement face 170, where the engagement face 170 of each insert half 110, 112 abuts the other.

In this exemplary embodiment, the insert halves 110, 112 cooperate to form a generally dome-shaped bearing surface 172. As will be discussed in more detail hereafter, alternative shaped bearing surfaces may be utilized such as, without limitation, flat horizontal surfaces, concave surfaces, sloped flat surfaces, and sloped arcuate surfaces. This bearing surface 172 provides a surface that rides upon the bearing surface(s) of the distal component 104. In this exemplary embodiment, the apex of the domed bearing surface 172 is proximate the engagement face 170, however, it is also within the scope of the disclosure to include an apex within the middle of the insert, posterior of the center of the insert, anterior of the center of the insert, lateral or medial of the center of the insert. In exemplary from, the insert halves comprise substantially mirror image halves 110, 112 that are joinable by way of a locking system.

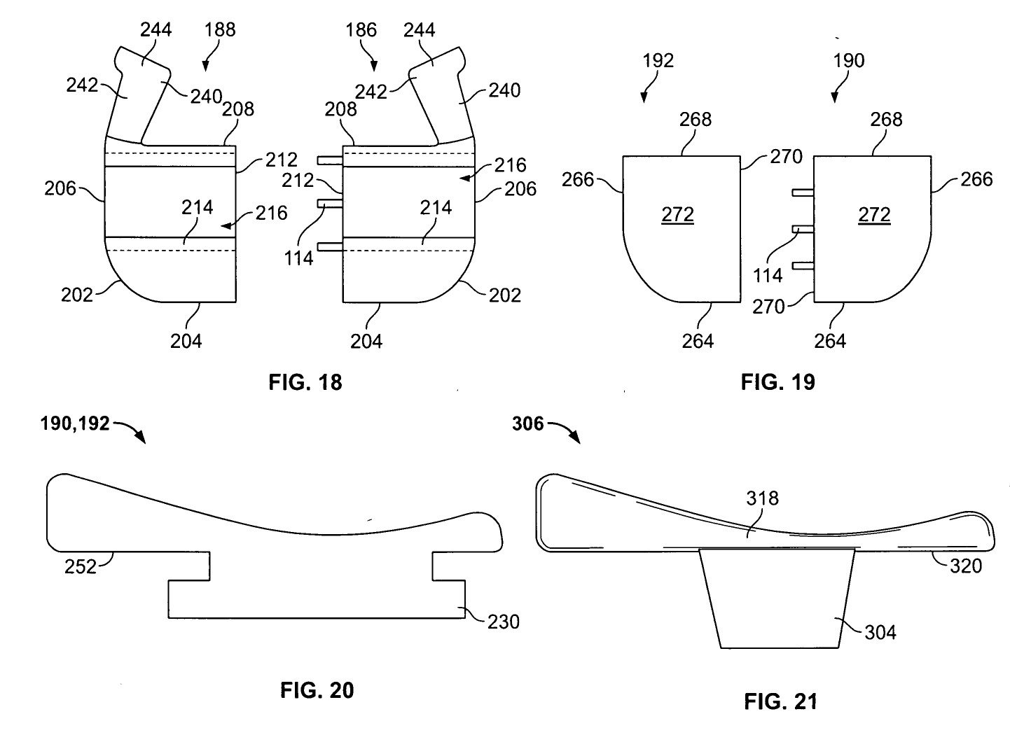

Referencing FIGS. 16-18, the exemplary distal component 104 includes a two-piece base plate 186, 188 and a two-piece insert 190, 192 mounted to the base plate 186, 188. In exemplary from, the base plate halves 186, 188 comprises substantially mirror image portions at are joinable by way of a locking system similar to that of the base plate 106, 108 of the proximal component 102.

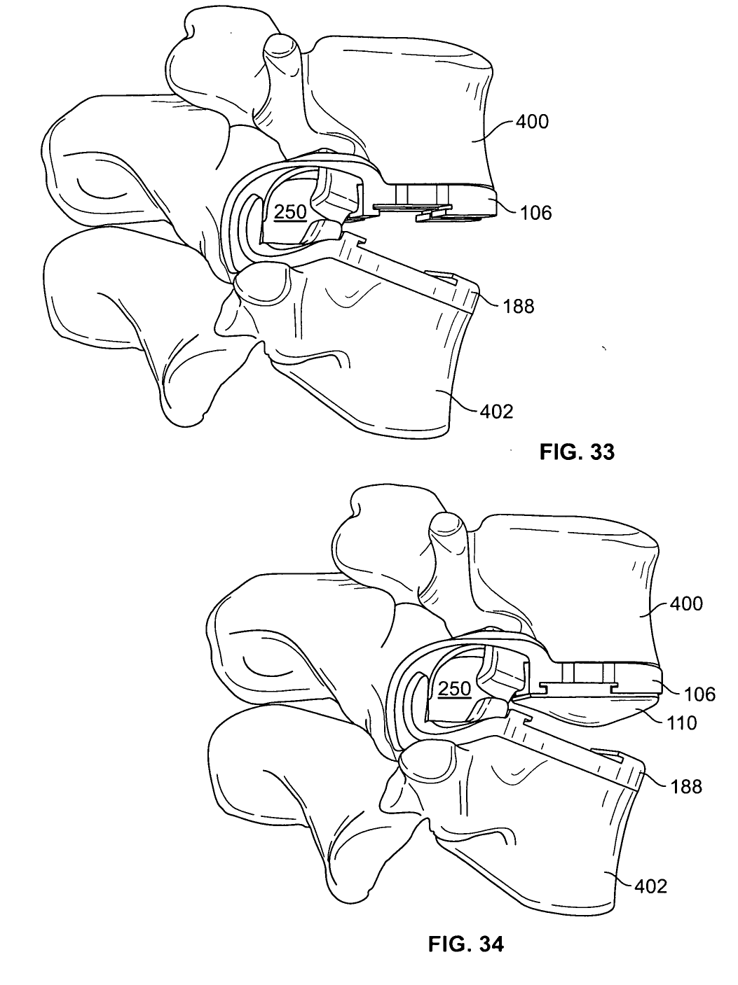

Each distal plate half 186, 188 includes a substantially planar bottom surface that is generally operative to cross-sectionally duplicate the vertebral body 22 (See e.g., FIGS. 32 and 33) against which it is mounted. For purposes of explanation only, each plate half 186, 188 includes this bottom surface and a continuous perimeter 202 comprising a front face 204 arcuately transitioning to a side face 206 that transitions into a rear face 208. Both the front face 204 and the rear face 208 transition at generally a right angle to an engagement face 212, where the engagement face 212 of each plate half 186, 188 abuts the other. A top surface 214 of each half 186, 188 includes a lateral groove 216 for receiving a T-shape projection 230 of one of the distal inserts 190, 192. In this exemplary embodiment, the groove 216 exhibits a T-shape cross-section to receive the T-shape projection 230 of the inserts 190, 192. It is also within the scope of the disclosure to utilize other shaped grooves such as, without limitation, dove-tail grooves, block grooves, and tapered grooves.

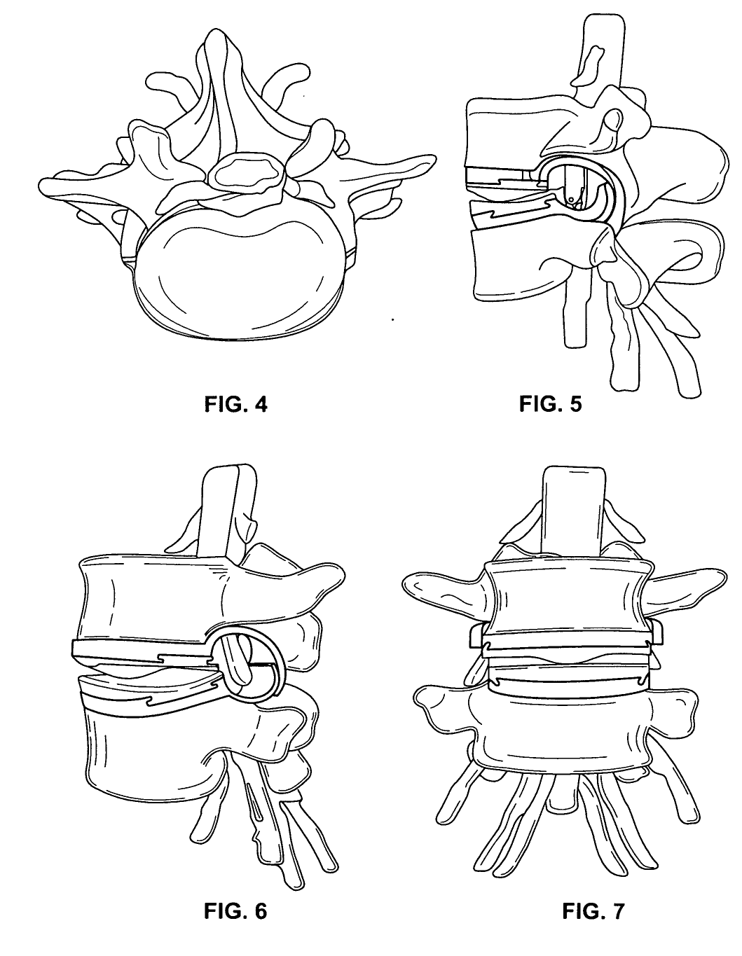

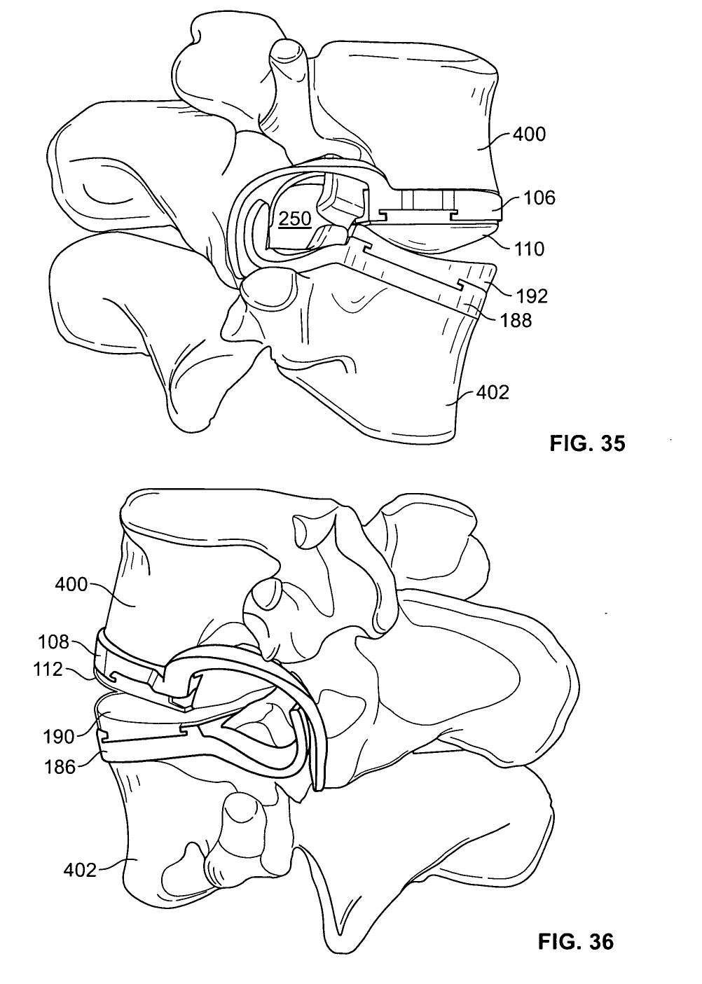

Each distal plate half 186, 188 also includes an arm 240 proximally extending from the rear face 208. Each arm 240, proximate the rear face 208, includes a rectangular cross-section (having front, back, and opposing sides), that transitions into a hood-shape with the width of the sides slightly decreasing and the width of the front and rear sides increasing. Each arm 240 exhibits a mild spiral 242 that terminates at a rounded end 244. The spiral 242 arcs rearward (away from the rear face 208) and inward (away from the side face 206) to terminate proximate the inferior processes 36 of the vertebrae above which it is mounted (see FIG. 5). The spiral 242 is adapted to cooperate with the spiral 152 of the proximal component 102 to define an opening 250 (see e.g., FIGS. 33-35) for egress of nerves exiting the spinal column between the vertebrae.

In exemplary form, plate halves 106, 108, 186, 188 may be fabricated from any surgical grade metal or metal alloy, plastic, ceramic, or any combination of the foregoing. In this first exemplary embodiment, however, the plate halves 106, 108, 186, 188 are fabricated from titanium or a titanium alloy.

Referring to FIGS. 19 and 20, each distal insert half 190, 192 cooperates to provide a bushing interposing the plate halves 186, 188 and the proximal component 102. As discussed briefly beforehand, each distal insert half 190, 192 includes a T-shape projection 230 that is received within a corresponding groove 216 of one of the distal plate halves 186, 188. This projection 230 extends from a substantially planar surface 252 that abuts a bottom surface of each distal plate half 186, 188. Depending upon the overall shape of the distal insert halves 190, 192, each half may exhibit a front face 264, arcuately transitioning to a side face 266 that transitions into a rear face 268. Both the front face 264 and the rear face 268 transition at generally a right angle to an engagement face 270, where the engagement face 270 of each insert half 190, 192 abuts the other when the halves are joined.

In this exemplary embodiment, the distal insert halves 190, 192 cooperate to form a concaved bearing surface 272. As will be discussed in more detail hereafter, alternative shaped bearing surfaces may be utilized such as, without limitation, flat horizontal surfaces, convex surfaces, sloped flat surfaces, and sloped arcuate surfaces. This bearing surface 272 provides a surface that rides against the bearing surface(s) of the proximal component 102. In exemplary from, the insert halves comprise substantially mirror image halves 190, 192 that are joinable by way of a locking system analogous to the locking system of the proximal insert halves 110, 112.

In exemplary form, the insert halves 110, 112, 190, 192 may be fabricated from any surgical grade metal or metal alloy, plastic, rubber, gel, ceramic, or any combination of the foregoing. In this first exemplary embodiment, however, the insert halves 110, 112, 190, 192 are fabricated from high molecular weight polyethylene.

The foregoing exemplary insert halves 110, 112, 190, 192 and plate halves 106, 108, 186, 188 include projections 114 and corresponding cavities 116 utilized to friction fit and lock the halves together in a proper orientation. Alternatively, the insert halves 110, 112, 190, 192 and plate halves may 106, 108, 186, 188 be formed together a unitary piece or each fabricated from two or more component parts. In addition, or in the alternative, the insert halves 110, 112, 190, 192 and plate halves 106, 108, 186, 188 may be cemented, glued, screwed, or otherwise fastened together.

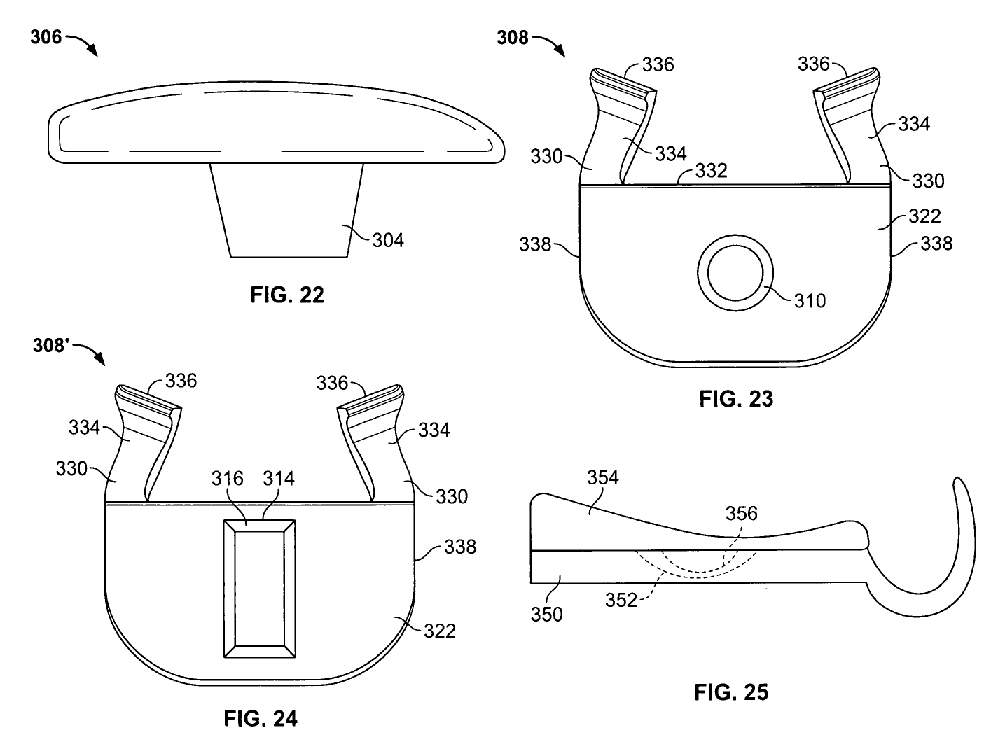

Referring to FIGS. 21-24, an alternate exemplary distal component includes a single piece insert 306 repositionably mounted to a single piece distal base plate 308. In this alternate exemplary component 304, the insert 306 includes a frustaconical projection 304 extending from its underside that is adapted to be received within cavity 310 formed within a top surface 312 of the distal base plate 308. In exemplary form, the cavity 310 may be frustaconical to allow for rotational movement of the insert 306 with respect to the base plate 308. In a further exemplary distal base plate 308′, the cavity comprises a longitudinal trench 314 extending partially from front to back and having sidewalls 316 that are tapered to approximate the angled nature of the frustaconical projection 304. This longitudinal trench 314 allows the insert 306 to move forward and backward, as well as rotate, thereby providing two degrees of movement.

Similar to the distal insert halves 190, 192 discussed as part of the first exemplary embodiment, the single piece insert 306 includes a dished bearing surface 318 on which a proximal insert would ride. The underside 320 is generally planar and adapted to sit upon a substantially planar top surface 322 of the distal base plate 308. Those skilled in the art will understand that the precise shape of the bearing surface 318 may be modified. For example, the bearing surface 318 may be flat horizontal surfaces, concave surfaces, sloped flat surfaces, and sloped arcuate surfaces.

Similar to the two-piece base plate 186, 188 halves discussed as part of the first exemplary embodiment, the single piece base plate 308 includes a pair of spaced apart arms 330 proximally extending from the rear face 332. Each arm 330, proximate the rear face 332, includes a rectangular cross-section (having front, back, and opposing sides), that transitions into a hood-shape with the width of the sides slightly decreasing and the width of the front and rear sides increasing. Each arm 330 exhibits a mild spiral 334 that terminates at a rounded end 336. The spiral 334 arcs rearward (away from the rear face 332) and inward (away from the side face 338) to terminate proximate the inferior processes 36 of the vertebrae above which it is mounted (see FIG. 5). The spiral 334 is adapted to cooperate with the spiral of a proximal component to define an opening (see e.g., FIGS. 33-35) for egress of nerves exiting the spinal column between the vertebrae.

Referring to FIG. 25, it is further within the scope of the disclosure for a base plate 350 to include a dished cavity 352. In such a circumstance, a bearing insert 354 may include a dome-shaped projection 356 that has a diameter and depth less than that of the dished cavity 352. In this alternate exemplary embodiment, the insert 354 has at least three degrees of freedom with respect to the base plate 350: (1) movement forward and backward; (2) movement from side to side; and, (3) rotational movement.

An exemplary surgical procedure for effectuating a spinal implant making use of one or more of the exemplary embodiments will now be discussed.

Initially, it is expected that the patient be placed prone on an operating table with all appropriate pressure points padded. Using C-arm fluoroscopy, both in anterior-posterior Ferguson and lateral directions, the pedicles immediately above and below the disc space are marked out on the skin surface with a linear pedicle-to-pedicle line. A linear skin incision is made from the pedicle-to-pedicle line as previously marked out. A K-wire is then passed to dock over the inferior articulating facet of the superior level, followed by insertion of a series of progressively larger tapered dilators that are operative provide an ever increasing exposure gap between otherwise adjacent muscle. A final visualization tube is then deployed to visualize the spinal facet joint, lateral lamina, and pars interarticularis. In exemplary form, the visualization tube is secured to a malleable arm attached to the operating table for holding the visualization tube in a secure position relative to the patient. Thereafter, anterior-posterior and lateral X-rays are taken to confirm the visualization tube placement.

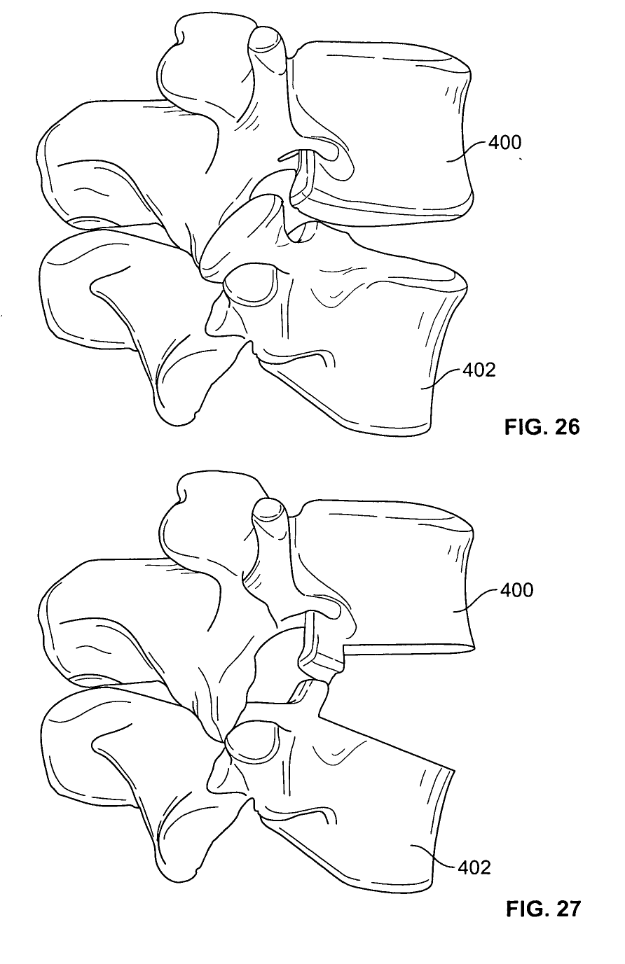

Referring to FIG. 26, with appropriate visualization and illumination (operating microscope, loupes, headlight, etc.) a drill, osteotome, or rongeur is used to resect the facet joint and the superior articulation of the inferior level to expose the disc space. After a complete facetectomy is completed, dissection is performed to expose the lateral dural edge, the traversing nerve root, and the disc space lateral thereto. A nerve root retractor is positioned and utilized to retract the dura and nerve medially and further expose the disk space laterally. A knife is thereafter used to create an annulotomy and initial discectomy is performed using rongeurs and scrapers. The interbody space is then sequentially dilated with in increasingly sized interbody spreaders under fluoroscopic guidance to assess the size of the disc space and total available height for the implant. The blunted edges of the spreaders will also decorticate the cartilaginous end plate, but leave the bony endplate of the vertebral bodies to eventually hold the implant. Any fragment of disc material not decorticated by the spreaders is removed with a pituitary rongeur or forceps.

Referencing FIG. 27, after the disc material is removed, the resulting vertebrae 400, 402 need to be prepared to accept the plate halves 106, 108, 186, 188. In exemplary form, a series of jigs (not shown) are mounted to the vertebrae in order to make horizontal and vertical cuts to form cut-outs within the anterior portion of the vertebral body for each vertebrae 400, 402. After the jigs are secured, a cutting tool is utilized to make straight horizontal and vertical cuts in to the vertebral body. Depending upon the precise jigs used, the vertical cuts may precede the horizontal cuts or vice versa. At the termination of the cutting, the vertebrae 400, 402 include generally planar horizontal adjacent surfaces that face one another. However, a portion of the posterior vertebral body is retained in order to provide a flange against which the plate halves 106, 108, 186, 188 are positioned. A series of trialers is thereafter used, to determine the appropriate implant size, followed by implantation of the artificial joint (implant).

Next, articulating trials (not shown) are used, under fluoroscopic imaging, to determine the appropriate implant size, the ideal thickness, and the ideal pivot point of the implant. If the subject, pre-operative, was compressed more medial, the pivot point would be made more medial to provide medial stability. If the subject, pre-operative, was compressed more lateral, the pivot point would be made more lateral to provide lateral stability. If the subject, pre-operative, was compressed more anterior, the pivot point would be made more anterior to provide anterior stability. If the subject, pre-operative, was compressed more posterior, the pivot point would be made more posterior to provide posterior stability. If the subject is restricted in internal/external rotation, then a mobile bearing articulating surface insert might be used to all greater rotational freedom. In addition, if the surgeon determines it is best for the subject have rotational and translational freedom, then a total freedom replacement could be used to allow the subject freely rotate and translated. Finally, if minimal rotation, translation or rotation and translation are required for a subject, then the articulating surface insert could have limited rotation and translation. Following trial fitting, implantation of the appropriate implant 100 may occur.

Referring to FIGS. 28-33, implantation of the artificial spinal joint includes initially placing the plate halves 106, 108, 186, 188 of the implant 100 into a holder (not shown) that includes a handle to be impacted with a mallet to place the plate halves into the disc space. Because the plate halves 106, 108, 186, 188 are without the polyethylene insert halves 110, 112, 190, 192 initially, it is easier to put into the plate halves into the disc space. In this exemplary embodiment, the proximal plate halves 106, 108 are positioned so that the rear face 140 abuts the flange of the posterior portion of the vertebral body of the vertebra 400, while the top surface 130 abuts the distal planar cut-out surface of the vertebral body. Similarly, the distal plate halves 186, 188 are positioned so that the rear face 208 abuts the flange of the posterior portion of the vertebral body of the vertebra 402, while the bottom planar surface abuts the proximal planar cut-out surface of the vertebral body. These plate halves 106, 108, 186, 188 are then impacted in a posterior oblique fashion under fluoroscopic guidance in order to position and lock the plate halves together using the locking system.

In exemplary form, the locking system may comprise two or more corresponding cavities 116 in the plate halves 106, 108, 186, 188 adapted to receive and retain a bolt 114 (threaded or smooth) operative to join and retain the corresponding plate halves. Alternatively, the locking system may include cavities in each of the plate halves 106, 108, 186, 188, that receive through rods to couple the plate halves together. Exemplary male and female retainers 114, 116 are well known to those skilled in the art. Specifically, the male projections 114 may include spline cylinders extending perpendicularly from the plate halves 106, 108, 186, 188 that are received within cylindrical openings 116 in the opposite/corresponding plate halves 106, 108, 186, 188 that are slightly smaller in diameter than the diameter of the spline cylinders. Exemplary locking systems may be fabricated from any combination of biologically acceptable materials including, without limitation, metals (titanium, stainless steel, etc.), ceramics, and plastics (high molecular weight polyethylene, polypropylene, etc.). Subsequent to the plate halves being positioned and locked together, the polyethylene insert halves 110, 112, 190, 192 are properly placed into the disc space.

Referring to FIGS. 34-36, each of the polyethylene insert halves 110, 112, 190, 192 are impacted into position to align each projection 160, 230 with its corresponding groove 146, 216 to position the insert halves within the center of each plate halve, thereby spreading the proximal plate halves 106, 108 from the distal plate halves 186, 188 to distract the disc space and restore height. Alternate shaped projections 160, 230 and grooves 146, 216 may be utilized so that the inserts can be simply impacted into the interspace between the plate halves 106, 108, 186, 188 in a posterior oblique fashion under fluoroscopic guidance so that the insert halves 110, 112, 190, 192 simply snap into the plate halves when the insert halves are in their proper position. In addition, the insert halves 110, 112, 190, 192 may include a separate locking system for securing the insert halves to one another.

In exemplary form, the locking system may comprise two or more corresponding cavities in the insert halves 110, 112, 190, 192 adapted to receive and retain a threaded bolt operative to join and retain the corresponding insert halves. Alternatively, the locking system may comprise one or more male projections 114 extending from one of the insert halves 110, 112, 190, 192, while the other corresponding insert halve 110, 112, 190, 192 includes corresponding female cavities 116 to receive the male projections so that coupled engagement occurs when the male projection is received within the female cavity. Exemplary male and female retainers are well known to those skilled in the art. Specifically, the male projections may include spline cylinders extending perpendicularly from the insert halves 110, 112, 190, 192 that are received within cylindrical openings in the opposite/corresponding insert halves 110, 112, 190, 192 that are slightly smaller in diameter than the diameter of the spline cylinders. Exemplary locking systems may be fabricated from any combination of biologically acceptable materials including, without limitation, metals (titanium, stainless steel, etc.), ceramics, and plastics (high molecular weight polyethylene, polypropylene, etc.).

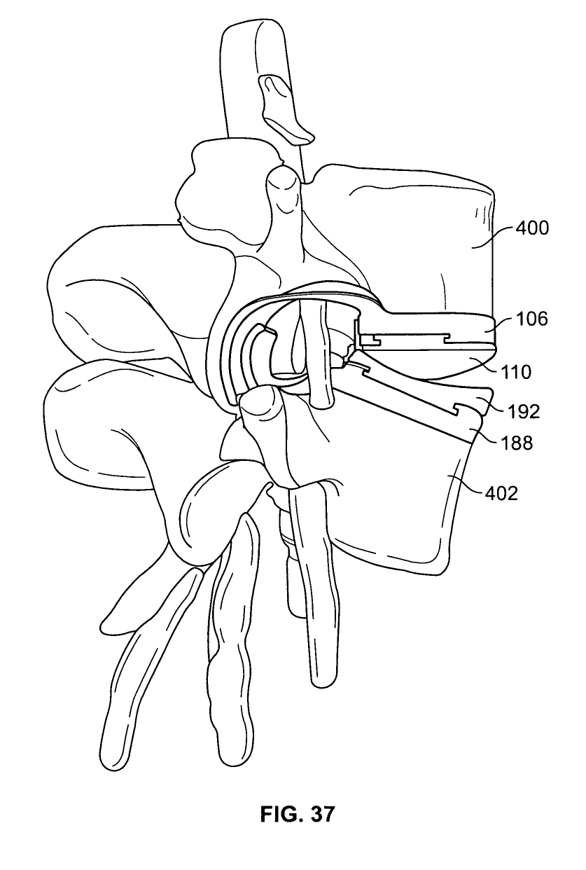

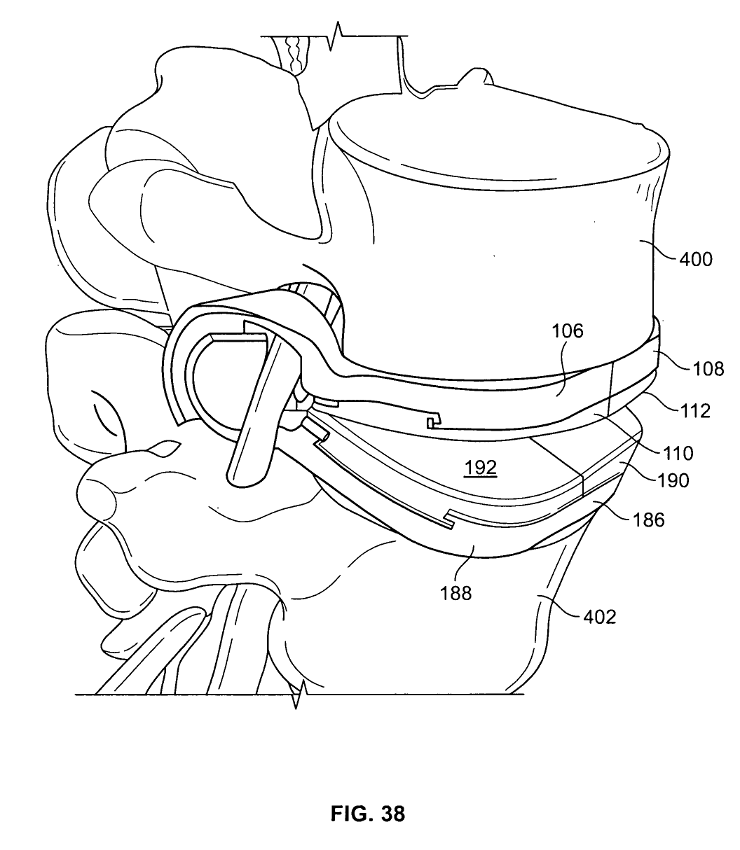

Referencing FIGS. 37 and 38, if any portion of the implant 100 is not in the correct position or if revision is needed, a handle (not shown) can be reattached to the plate halves or insert halves to facilitate reposition or revision. Once the final placement of the implant is completed, a vertebral body screw (not shown) is placed into the eyelets of both plates to hole them in place. However, these screws have cancellous purchase and are not strong enough to permanently hold the implants in place by themselves. A facet joint attachment is then chosen based on the distance between the holding threads of the plate halves 106, 108, 186, 188, which has an opening for placement of a pedicle screw which is strong enough to hold the implant in place permanently. This joint connects to the plate halves 106, 108, 186, 188 by the holding threads, screwed on, and different sizes allow for anatomic variations of the pedicle to disc space distance. Final tightening is then performed of all screws with a torque wrench. The wound is then inspected, with decompression of the neural elements verified using a Woodson dental tool or other surgical probe.

In an alternate exemplary embodiment, the bottom surfaces 131 of the plate halves 106, 108, 186, 188 may be cemented or press-fit onto the adjacent vertebrae bone. Alternatively, the halves may be mounted to the adjacent vertebrae bone using surgical screws or other fastening devices. It is also within the scope of the present disclosure to incorporate a bottom surface 131 of the plate halves 106, 108, 186, 188 that is textured and provides for an improved bonding surface when using at least one of cement and bone ingrowth material.

While the foregoing exemplary embodiment has been described with the inserts 110, 112, 190, 192 including a projection that is received within a groove of one of the plate halves 106, 108, 186, 188, it is also within the scope of the disclosure for the plate halves to each include a projection that is received within a groove of one of the inserts 110, 112, 190, 192. Those skilled in the art will certainly understand the interchangeability of the groove 146, 216 and corresponding projection 160, 230 in the foregoing exemplary embodiments. In addition, the grooves 146, 216 need not be oriented laterally, but instead may be oriented from front to back.

In exemplary form, the lumbar spinal implant 100 is sized or scaled to match a patient's vertebrae. In other words, while the general shape of an L5 vertebra, for example, is the same for human beings, anatomical considerations and range of motion for each patient warrant sizing or scaling the implant to match the vertebrae of the patient. Likewise, the spinal implants 100 are shaped according to the location of the disc replacement. Because the disc size and, shape of a cervical disc is not identical to the size and shape of a lumbar disc within the same patient, the exemplary implants may be sized and shaped based upon the intended location of use. Moreover, the articulating surfaces may be sized to be gender and/or ethnic specific.

By way of example, and not limitation, the present embodiments are also amendable for future revision surgery where one or more of the inserts would be replaced without replacing the base plate halves. In this manner, minimally invasive surgery would be an practical alternative to replace the bearing inserts.

It is also within the scope of the disclosure that the base plates be fabricated from only one or more than two component parts. While some of the foregoing embodiments show base plates comprising complementary halves, these same components may be fabricated as a unitary structure or fabricated from three or more pieces, not necessarily substantially the same size. Along these same lines, the base plates may vary in thickness (distance between top and bottom surfaces) depending upon the disc to be replaced.

It is also within the scope of the present disclosure for the distal inserts 190, 192 to exhibit a domed shape, while the proximal inserts 110, 112 exhibit a concave shape. Along these same lines, the thickness and other shape features are interchangeable between the proximal and distal inserts of the present disclosure.

If each base plate is one piece, it can be attached to the bone either from the lateral direction, medial direction and/or the frontal direction. Alternatively, if the base plate is two or three pieces, it could be attached to the bone from the lateral direction, medial direction and/or the frontal direction or from multiple directions.

It is further within the scope of the disclosure for the respective base plate halves and insert halves to be fabricated as a unitary structure. In other words, the base plate would embody the any mobile bearing features of the insert and the vertebra bone would be contoured to accommodate this increased range of motion.

Following from the above description and disclosure summaries, it should be apparent to those of ordinary skill in the art that, while the methods and apparatuses herein described constitute exemplary embodiments of the present invention, the inventions disclosed herein are not limited to any precise embodiment and that changes may be made to any such embodiment without departing from the scope of the invention as defined by the claims. Additionally, it is to be understood that the invention is defined by the claims and it is not intended that any limitations or elements describing the exemplary embodiments set forth herein are to be incorporated into the interpretation of any claim element unless such limitation or element is explicitly stated. Likewise, it is to be understood that it is not necessary to meet any or all of the identified advantages or objects of the invention disclosed herein in order to fall within the scope of any claims, since the invention is defined by the claims and since inherent and/or unforeseen advantages of the present invention may exist even though they may not have been explicitly discussed herein.

What is claimed is:

1. A spinal disc prosthesis comprising:
a distal spinal prosthetic comprising at least two separable portions; and
a proximal spinal prosthetic comprising at least two separable portions;
where at least one of the two separable portions of at least one of the distal spinal prosthetic and the proximal spinal prosthetic includes a partial loop appendage at least partially defining a spinal nerve canal.

2. The spinal disc prosthesis of claim 1, wherein the distal spinal prosthetic includes a first bearing insert mounted to at least one of the at least two separable portions, the first bearing insert including a bearing surface interposing the distal spinal prosthetic and the proximal spinal prosthetic.

3. The spinal disc prosthesis of claim 2, wherein at least one of the complementary halves includes a cavity for receiving a projection of the first bearing insert to mount the first bearing insert to at least one of the at least two separable portions.

4. The spinal disc prosthesis of claim 2, wherein at least one of the complementary halves includes a projection received within a cavity of the first bearing insert to mount the first bearing insert to at least one of the at least two separable portions.

5. The spinal disc prosthesis of claim 1, wherein the partial loop appendage extends from a rear surface of at least one of the distal spinal prosthetic and the proximal spinal prosthetic.

6. The spinal disc prosthesis of claim 1, wherein the partial loop appendage extends from a top surface of at least one of the distal spinal prosthetic and the proximal spinal prosthetic.

7. The spinal disc prosthesis of claim 1, wherein the partial loop appendage extends from a bottom surface of at least one of the distal spinal prosthetic and the proximal spinal prosthetic.

8. The spinal disc prosthesis of claim 1, wherein the partial loop appendage extends from a side surface of at least one of the distal spinal prosthetic and the proximal spinal prosthetic.

9. The spinal disc prosthesis of claim 1, wherein the partial loop appendage includes a helical portion.

10. The spinal disc prosthesis of claim 1, wherein both the distal spinal prosthetic and the proximal spinal prosthetic include at least one partial loop appendage at least partially defining the spinal nerve canal.

11. The spinal disc prosthesis of claim 10, wherein both the distal spinal prosthetic and the proximal spinal prosthetic include a pair of partial loop appendages at least partially defining the spinal nerve canal.

12. The spinal disc prosthesis of claim 10, wherein the pair of partial loop appendages of the proximal spinal prosthetic includes an arcuate diameter less than an arcuate diameter of the pair of partial loop appendages of the distal spinal prosthetic.

13. The spinal disc prosthesis of claim 10, wherein interior surfaces of the pair of partial loop appendages of the distal spinal prosthetic provides camming surfaces for exterior surfaces of the pair of partial loop appendages of the proximal spinal prosthetic.

14. The spinal disc prosthesis of claim 1, wherein the distal spinal prosthetic includes a pair of partial loop appendages at least partially defining the spinal nerve canal.

15. The spinal disc prosthesis of claim 1, wherein the proximally spinal prosthetic includes a pair of partial loop appendages at least partially defining the spinal nerve canal.

16. The spinal disc prosthesis of claim 1, wherein the partial loop appendage includes a facet replacement.

17. The spinal disc prosthesis of claim 1, wherein the proximal spinal prosthetic includes a first bearing insert mounted to at least one of the at least two separable portions, the first bearing insert including a bearing surface interposing the distal spinal prosthetic and the proximal spinal prosthetic.

18. The spinal disc prosthesis of claim 17, wherein at least one of the complementary halves includes a cavity for receiving a projection of the first bearing insert to mount the first bearing insert to at least one of the at least two separable portions.

19. The spinal disc prosthesis of claim 17, wherein at least one of the complementary halves includes a projection received within a cavity of the first bearing insert to mount the first bearing insert to at least one of the at least two separable portions.

20. The spinal disc prosthesis of claim 17, wherein at least one of the distal spinal prosthetic and the proximal spinal prosthetic includes a twisted appendage including a facet interface pad.

21. The spinal disc prosthesis of claim 2, wherein the first bearing insert is a fixed bearing insert with respect to at least one of the at least two separable portions.

22. The spinal disc prosthesis of claim 2, wherein the first bearing insert is a mobile bearing insert with respect to at least one of the at least two separable portions.

23. An intervertebral prosthetic comprising:
a first spinal implant including a vertebral body portion, the vertebral body portion at least partially replicating the horizontal cross-section of a vertebral body and including a vertebral contacting surface adapted to contact a native vertebral body, the first spinal implant also including a disc portion including a bearing surface, the first spinal implant also including an appendage at least partially defining a spinal nerve conduit.

24. The intervertebral prosthetic of claim 23, wherein the first implant includes a pair of appendages at least partially defining a spinal nerve conduit.

25. The intervertebral prosthetic of claim 23, wherein the appendage includes a helical portion.

26. The intervertebral prosthetic of claim 23, wherein the appendage includes a twisted portion.

27. The intervertebral prosthetic of claim 23, wherein the appendage includes a facet replacement portion.

28. The intervertebral prosthetic of claim 23, wherein the vertebral body portion is a separable component from the disc portion.

29. The intervertebral prosthetic of claim 28, wherein the vertebral body portion is fabricated from a metal and the disc portion is fabricated from a polymer.

30. The intervertebral prosthetic of claim 28, wherein the vertebral body portion includes a cavity that receives a projection of the disc portion to mount the vertebral body portion to the disc portion.

31. The intervertebral prosthetic of claim 28, wherein the disc portion includes a cavity that receives a projection of the vertebral body portion to mount the vertebral body portion to the disc portion.

32. The intervertebral prosthetic of claim 28, wherein the vertebral body portion is at least one of glued and cemented to the disc portion.

33. An intervertebral implant comprising:
a first implant adapted to mate with a first vertebra, where the first implant includes an arm operative to at least partially define a spinal nerve passage; and
a second implant adapted to mate with a second vertebra consecutive to the first vertebra, where the second implant includes an appendage operative to cooperate with the arm of the first implant to at least partially define the spinal nerve passage.

34. The intervertebral implant of claim 33, wherein the first implant includes a first bearing insert having a first bearing surface interposing the first vertebra and the second vertebra.

35. The intervertebral implant of claim 34, wherein the second implant includes a second bearing insert having a second bearing surface interposing the first vertebra and the second vertebra, the second bearing surface adapted to interface with the first bearing surface of the first implant.

36. The intervertebral implant of claim 33, wherein the first implant comprises a first vertebral segment and a first disc segment mounted to one another.

37. The intervertebral implant of claim 36, wherein the first vertebral segment includes a cavity receiving a projection from the first disc segment to mount the first vertebral segment to the first disc segment.

38. The intervertebral implant of claim 36, wherein the first disc segment includes a cavity receiving a projection from the first vertebral segment to mount the first vertebral segment to the first disc segment.

39. The intervertebral implant of claim 33, wherein the first implant comprises at least two component parts simulating the horizontal cross-section of a vertebral body.

40. The intervertebral implant of claim 39, wherein the at least two component parts comprise complementary halves of a base plate.

41. The intervertebral implant of claim 40, wherein the base plate halves are mounted to a bearing insert.

42. The intervertebral implant of claim 33, wherein the second implant comprises a second vertebral segment and a second disc segment mounted to one another.

43. The intervertebral implant of claim 42, wherein the first vertebral segment includes a cavity receiving a projection from the first disc segment to mount the first vertebral segment to the first disc segment.

44. The intervertebral implant of claim 42, wherein the first disc segment includes a cavity receiving a projection from the first vertebral segment to mount the first vertebral segment to the first disc segment.

43. The intervertebral implant of claim 33, wherein the second implant comprises at least two component parts simulating the horizontal cross-section of a vertebral body.

44. The intervertebral implant of claim 43, wherein the at least two component parts comprise complementary halves of a base plate.

45. The intervertebral implant of claim 44, wherein the base plate halves are adapted to receive a bearing insert.

46. The intervertebral implant of claim 33, wherein the arm of the first implant extends from a rear surface a base plate at least partially simulating the horizontal cross-section of a vertebral body.

47. The intervertebral implant of claim 33, wherein the arm of the first implant extends from a top surface a base plate at least partially simulating the horizontal cross-section of a vertebral body.

48. The intervertebral implant of claim 33, wherein the arm of the first implant extends from a bottom surface a base plate at least partially simulating the horizontal cross-section of a vertebral body.

49. The intervertebral implant of claim 33, wherein the arm of the first implant extends from a side surface a base plate at least partially simulating the horizontal cross-section of a vertebral body.

50. The intervertebral implant of claim 33, wherein the arm of the first implant includes a helical portion.

51. The intervertebral implant of claim 33, wherein the appendage of the second implant extends from a rear surface a base plate at least partially simulating the horizontal cross-section of a vertebral body.

52. The intervertebral implant of claim 33, wherein the appendage of the second implant extends from a top surface a base plate at least partially simulating the horizontal cross-section of a vertebral body.

53. The intervertebral implant of claim 33, wherein the appendage of the second implant extends from a bottom surface a base plate at least partially simulating the horizontal cross-section of a vertebral body.

54. The intervertebral implant of claim 33, wherein the appendage of the second implant extends from a side surface a base plate at least partially simulating the horizontal cross-section of a vertebral body.

55. The intervertebral implant of claim 33, wherein the appendage of the second implant includes a helical portion.

56. The intervertebral implant of claim 33, wherein:
the first implant includes a pair of arms operative to at least partially define a pair of spinal nerve passages; and
the second implant includes a pair of appendages operative to at least partially define the pair of spinal nerve passages.

57. The intervertebral implant of claim 56, wherein:
each of the pair of arms includes at least one of a twisted portion and a helical portion; and
each of the pair of appendages includes at least one of a twisted portion and a helical portion.

58. The intervertebral implant of claim 57, wherein:
each of the pair of arms includes a helical portion;
each of the pair of appendages includes a helical portion; and
each of the pair of arms of the first implant includes an arcuate diameter less than an arcuate diameter of the pair of appendages of the second implant.

59. The intervertebral implant of claim 33, wherein the arm includes a facet replacement.

60. The intervertebral implant of claim 33, wherein the appendage includes a facet replacement.

61. A spinal implant comprising:
a first platform allowing fixation to a first vertebra;
a second platform allowing fixation to a second vertebra immediately following the first vertebra; and
a bearing interposing the first platform and the second platform;
wherein at least one of the first platform, the second platform, and the bearing includes an arcuate projection at least partially defining a spinal nerve passageway.

62. A spinal disc prosthesis comprising:
a distal spinal prosthetic comprising a distal base plate and a distal bearing; and
a proximal spinal prosthetic comprising a proximal base plate and a proximal bearing;
where at least one of the distal spinal prosthetic and the proximal spinal prosthetic includes an appendage at least partially defining a spinal nerve canal.
